(19) United States
(12) Patent Application Publication
(10) Pub. No.: US 2005/0171537 A1
(43) Pub. Date: Aug. 4, 2005

(54) Connector for vertebral anchoring system

(76) Inventors: Christian Mazel, Vaucresson (FR); Eric Leroy, Saint Nicolas (FR); Jean-Yves Leroy, Campagne Les-Hesdin (FR); Pommier Arnaud, Raimbeaucourt (FR)

Correspondence Address: YOUNG & THOMPSON, 745 SOUTH 23RD STREET, 2ND FLOOR, ARLINGTON, VA, 22202, US

(21) Appl. No.: 10/496,688

(22) PCT Filed: Nov. 19, 2002
(86) PCT No.: PCT/FR02/03951

(30) Foreign Application Priority Data
Nov. 27, 2001 (FR) 01/15298

(51) Int. Cl. A61B 17/58

(52) U.S. Cl. 606/61

(57) ABSTRACT

Each connector of the vertebral anchoring system for spinal fixation includes: a first element having parallel housings in cylinder portion receiving respectively the corresponding linking rod and a threaded hollow cylindrical sleeve extending vertically between the housings; a second element having parallel housings in cylinder portion receiving respectively the corresponding linking rod and a bore provided between the housings to be traversed by the cylindrical sleeve of the first element; a clamping nut co-operating with a male thread of the cylindrical sleeve of the first element for fixing the second element on the first and the linking rods in the housings; and clamping elements co-operating with the cylindrical sleeve of the first element for fixing each connector on the corresponding anchoring screw.

### The present invention relates to a connector for a vertebral anchoring system permitting the use either of a multi-axial bone-anchoring screw with spherical head or of a bone-anchoring screw with threaded head.

U.S. Pat. No. 3 997 138 discloses vertebral anchoring systems for spinal osteosynthesis, having fixation screws which consist of a threaded part for bone anchoring and of a hexagonal drive head which, at the end remote from the first threaded part, is continued by a second threaded part with mechanical pitch.

The hexagonal head of the fixation screw has two parallel channels arranged on either side of the second threaded part in order to receive a respective connection rod.

The vertebral anchoring system includes a retention element of hexagonal shape which is traversed by a central bore, cooperating with the second threaded part of the fixation screw, and by two parallel channels arranged on either side of the central bore.

The channels of the retention element are positioned in such a way as to cooperate with those of the head of the fixation screw.

The vertebral anchoring system includes a clamping nut of hexagonal profile which cooperates with the second threaded part of the anchoring screw in order, on the one hand, to immobilize the retention element on the head of the screw, and, on the other hand, to immobilize the connection rods in translation and rotation inside parallel channels.

It will be noted that the vertebral anchoring system does not permit angular adjustment of the retention element and, consequently, of the connection rods relative to the fixation screw.

Also, this type of anchoring system is designed exclusively for fixing on fixation screws with threaded head and it cannot receive other fixation screws, such as those with spherical head or multi-axial head.

The vertebral anchoring system according to the present invention is intended to provide connectors which are able to receive either multi-axial screws or screws with a threaded head, and to permit angular adjustment about the head of the corresponding screw.

The vertebral anchoring system for spinal osteosynthesis according to the present invention has connectors which each consist of:
a first element comprising parallel seats in the shape of a segment of a cylinder, each receiving the corresponding connection rod, and a threaded hollow cylindrical sleeve extending vertically between said seats,
a second element comprising parallel seats in the shape of a segment of a cylinder, each receiving the corresponding connection rod, and a bore arranged between said seats so as to be traversed by the cylindrical sleeve of the first element,
a clamping nut cooperating with an external thread of the cylindrical sleeve of the first element in order, on the one hand, to fix the second element on the first, and, on the other hand, to fix the connection rods in the seats,
and clamping means cooperating with the cylindrical sleeve of the first element for fixing each connector on the corresponding vertebral anchoring screw.

The vertebral anchoring system for spinal osteosynthesis according to the present invention has a cylindrical sleeve which, in its inner part, has a through-bore provided with a thread, a first surface in the shape of a segment of a sphere, and a second surface of conical profile.

The vertebral anchoring system for spinal osteosynthesis according to the present invention has a cylindrical sleeve whose internal thread cooperates with the clamping means upon fixation of each connector on a multi-axial anchoring screw.

The vertebral anchoring system for spinal osteosynthesis according to the present invention has a cylindrical sleeve whose spherical surface is continued, in the direction of the outside of said sleeve, by a second surface of conical profile whose widest base is oriented in the direction of the outside of the first element.

The vertebral anchoring system for spinal osteosynthesis according to the present invention has a cylindrical sleeve whose spherical surface has, at its base, a shoulder separating the latter from the conical surface, so as to constitute a support or bearing face for the corresponding fixation screw.

The vertebral anchoring system for spinal osteosynthesis according to the present invention has a cylindrical sleeve whose upper part, forming the free end, has a plane face forming a bearing face for the clamping means upon fixation of each connector on an anchoring screw with threaded head.

The vertebral anchoring system for spinal osteosynthesis according to the present invention has clamping means which consist of a threaded plug with internal profile permitting fixation of each connector on multi-axial anchoring screws with spherical head.

The vertebral anchoring system for spinal osteosynthesis according to the present invention has clamping means which consist of a clamping nut with external profile permitting fixation of each connector on anchoring screws with threaded head.

The vertebral anchoring system for spinal osteosynthesis according to the present invention has a cylindrical sleeve provided, on its outer face and over its entire height, with two parallel flat surfaces.

The following description in which reference is made to the attached drawings, given as non-limiting examples, will permit a better understanding of the invention, its characteristics, and the advantages it may afford.

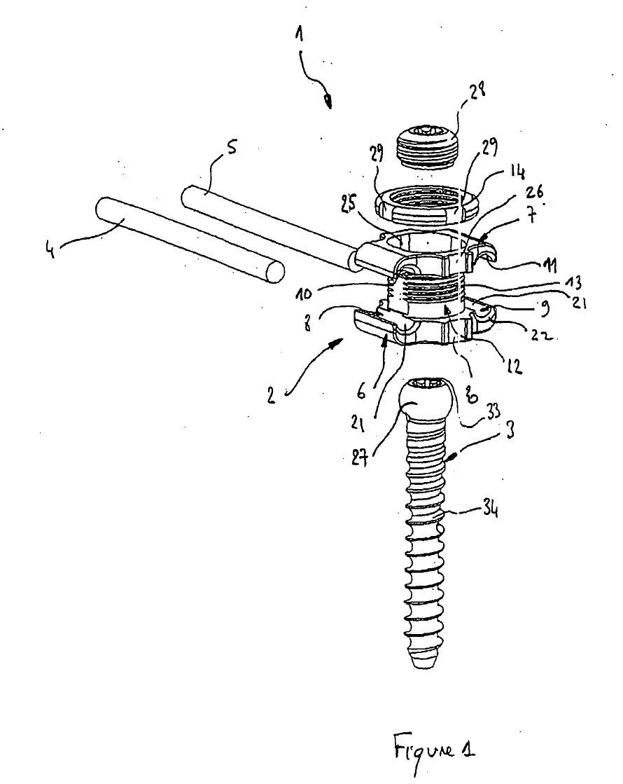

FIG. 1 is an exploded perspective view illustrating a connector for a vertebral anchoring system with multi-axial screw according to the present invention.

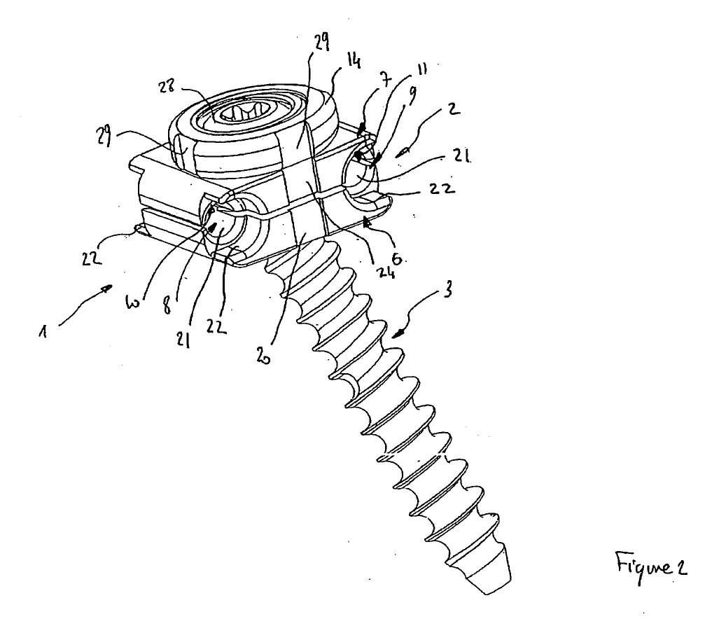

FIG. 2 is a perspective view showing the connector for a vertebral anchoring system with multi-axial screw in the assembled position.

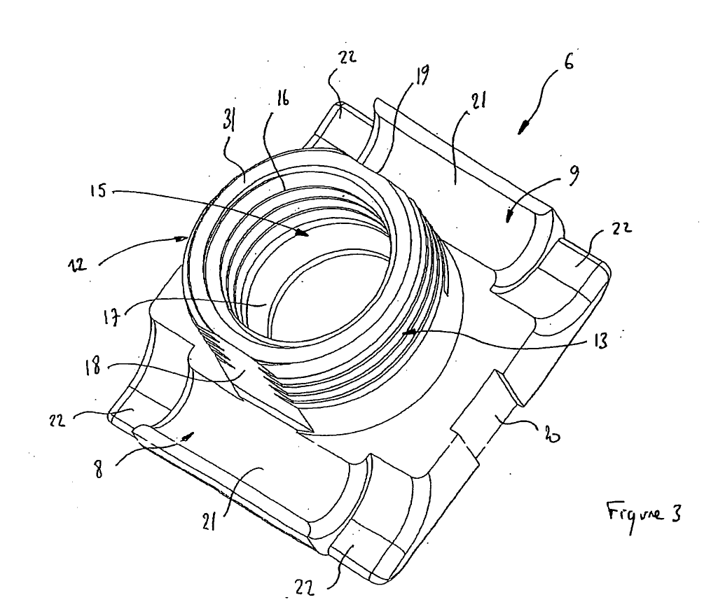

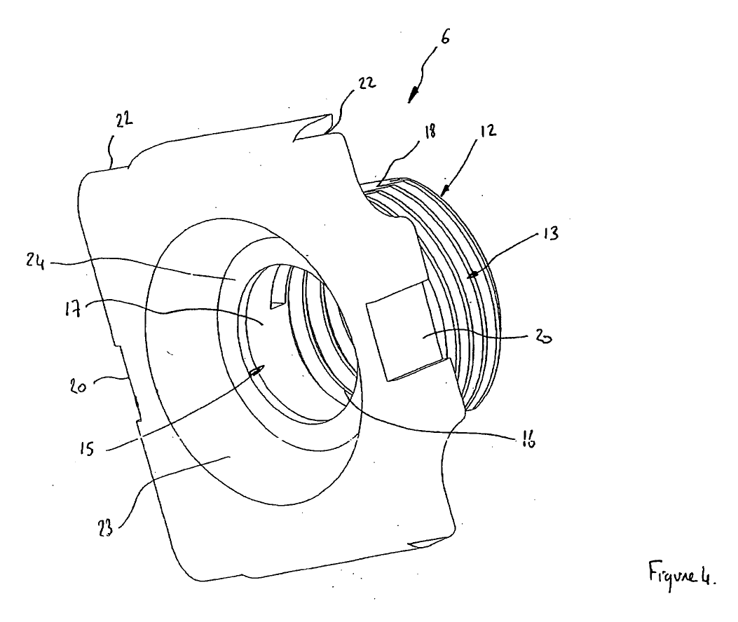

FIGS. 3 and 4 are perspective views showing in detail one of the elements of the connector for a vertebral anchoring system according to the present invention.

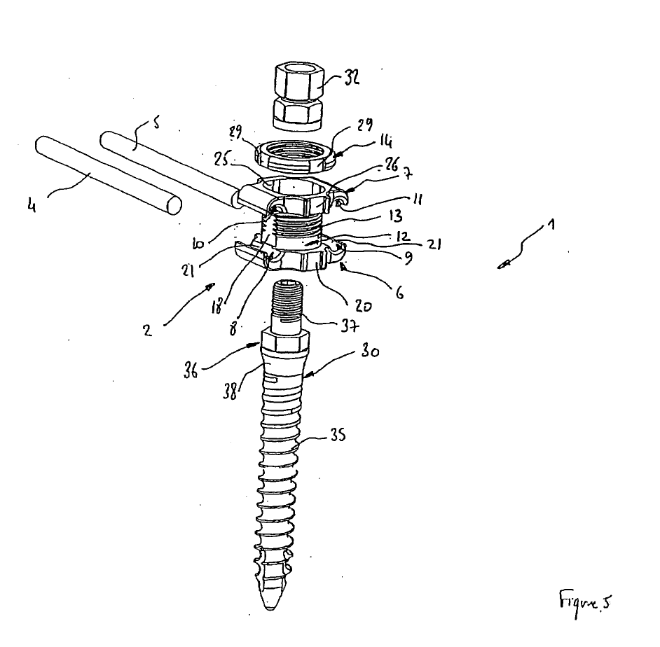

FIG. 5 is an exploded perspective view showing a connector for a vertebral anchoring system with double-anchor screw according to the present invention.

In FIGS. 1 to 5, a vertebral anchoring system 1 has been shown which includes a connector 2 intended to cooperate with a multi-axial screw 3 or a screw with threaded head 30 in order to permit immobilization of two connection rods 4 and 5.

The connector 2 consists of a first, lower element 6 cooperating with a second, upper element 7, each provided with seats 8, 9, 10 and 11 in the shape of a segment of a cylinder for receiving and immobilizing the connection rods 4 and 5 by means of a clamping nut 14.

FIGS. 3 and 4 show the first element 6 of the connector 2 which consists of a hollow cylindrical sleeve 12 extending in a vertical direction and perpendicular to the seats 8 and 9.

The seats 8 and 9 in the shape of a segment of a cylinder belonging to the element 6 are formed on either side of the cylindrical sleeve 12 and parallel to one another.

The hollow cylindrical sleeve 12 has, on its outer face, a thread 13 which, upon assembly of the anchoring system 1, cooperates with the clamping nut 14 in order, on the one hand, to immobilize the second element 7 on the first 6 and, on the other hand, to immobilize the connection rods 4 and 5 in the seats 8, 9, 10 and 11 (FIG. 2).

The clamping nut 14 has, on its outer circumference, notches 29 allowing it to be driven in rotation around the external thread 13 of the sleeve 12.

The cylindrical sleeve 12 has, in its inner part, a through-bore 15 provided with a thread 16, a first surface 17 in the shape of a segment of a sphere, and a second surface 23 of conical profile.

In the case where a multi-axial screw 3 is used, the internal thread 16 cooperates with a threaded plug 28 with internal profile permitting immobilization and locking in a defined position of said screw 3 (FIG. 2).

The spherical surface 17 is continued, in the direction of the outside of the cylindrical sleeve 12, by the second surface 23 of conical profile whose widest base is oriented in the direction of the outside of the element 6.

The spherical surface 17 and conical surface 23 are positioned at the base of the cylindrical sleeve 12, that is to say in the area of the seats 8 and 9 formed as segments of a circle in the element 6.

The spherical surface 17 has at its base, that is to say at the end remote from the internal thread 16, a shoulder 24 which separates the latter from the conical surface 23, so as to constitute a support or bearing face for the fixation screw 3 and 30.

The cylindrical sleeve 12 has, on its outer face and over its entire height, two parallel flat surfaces 18 and 19, so as to truncate the thread 13.

The two flat surfaces 18 and 19 make it possible to reduce the external diameter of the cylindrical sleeve 12 in order to avoid the latter protruding into the seats 8 and 9 in the shape of a segment of a cylinder belonging to the element 6.

The upper part forming the free end of the cylindrical sleeve 12 has a plane face 31 serving as a support for a clamping nut 32 with external recesses when a screw with threaded head 30 is used (FIG. 5).

The first element 6 has, between the seats 8 and 9 in the shape of segments of a cylinder, recesses 20 positioned in a vertical direction and parallel to the main axis of the internal bore 15 of the sleeve 12.

The seats 8 and 9 have a support 21 in the shape of a segment of a cylinder which is delimited at each end by shoulders 22 situated below the profile, in the shape of a segment of a circle, of the support 21.

Thus, the supports 21 in the shape of segments of a cylinder for each seat 8 and 9 have a length which is less than that of the element 6 in order to guarantee positioning and perfect immobilization of the connection rods 4 and 5 over a sufficient distance.

Between the seats 10 and 11 in the shape of segments of a cylinder, the second element 7 has a bore 25 which is traversed by the cylindrical sleeve 12 of the first element 6 (FIG. 1).

Between the seats 10 and 11 in the shape of segments of a cylinder, the second element 7 has recesses 26 positioned in a vertical direction and parallel to the main axis of the bore 25 (FIG. 1).

The seats 10 and 11 are similar to those 8 and 9 of the first element 6 in such a way as to present a support in the shape of a segment of a cylinder and which is delimited, at each end, by shoulders situated below the profile, in the shape of a segment of a cylinder, of the support.

The seats 8, 9, 10 and 11 of each element 6, 7 are intended to receive the connection rods 4 and 5, respectively, so as to be able to position them and immobilize them in rotation and translation inside each element 6, 7 of the connector 2 upon clamping of the nut 14 on the sleeve 12.

In FIG. 2, the connector 2 according to the present invention has been illustrated in the assembled and immobilized state on a multi-axial screw 3.

The multi-axial screw 3 has a spherical head 27 provided with an internal profile 33, for driving said screw in rotation and anchoring it in the corresponding vertebral body.

The spherical head 27 of the multi-axial screw 3 is continued by a threaded body 34, whose thread progresses and cuts in the direction toward the tip of said screw.

Before being anchored in the vertebral body, the multi-axial screw 3 is introduced into the internal bore 15 of the cylindrical sleeve 12 of the first element 6, so that its spherical head 27 comes to bear on the face of the support 24 which is directed toward the spherical surface 17.

After the screw 3 has been anchored in the corresponding vertebral body, the second element 7 is immobilized on the first one 6 with the aid of the nut 14 which cooperates with the external thread 13 of the sleeve 12. The immobilization of the second element 7 at the same time effects locking of the connection rods 4 and 5 in the corresponding seats 8, 9, 10 and 11 of the connector 2.

The conical surface 24 provided in the internal bore 15 of the cylindrical sleeve 12 permits angular adjustment of the connector 2 about the spherical head 27 of the multi-axial screw 3.

The connector 2 is immobilized in its angular position about the head 27 of the multi-axial screw by way of the plug 28 which is screwed inside the cylindrical sleeve 12.

In FIG. 5, the connector 2 according to the present invention is shown mounted on a screw with threaded head 30.

The screw 30 has a first threaded bone-anchoring part 35, a drive head 36 and, in the continuation of the head, a second threaded part 37 which cooperates with a clamping nut 32.

The drive head 36 has a smooth portion of flared profile 38 situated between the threaded bone-anchoring part 35 and said head. The latter has a hexagonal external profile for driving the screw 30 in rotation.

The screw with threaded head 30 is anchored in the corresponding vertebral body before receiving the first element 6 of the connector 2. The first element 6 is positioned on the screw 30 in such a way that the head 36 comes to bear on the face of the shoulder 24 directed toward the conical surface 23 formed in the cylindrical sleeve 12.

The second element 7 is then immobilized on the first 6 with the aid of the nut 14 which cooperates with the external thread 13 of the sleeve 12. The immobilization of the second element 7 at the same time effects blocking of the connection rods 4 and 5 in the corresponding seats 8, 9, 10 and 11 of the connector 2.

The connector 2 is immobilized on the screw 30 by way of the clamping nut 32 which is screwed onto the second threaded part 37 until it is blocked against the plane face 31 of the cylindrical sleeve 12.

It must also be appreciated that the above description has been given only by way of example and that it does not in any way limit the scope of the invention, and that replacing the described details with any other equivalents would not constitute a departure from the scope of the invention.

1. A vertebral anchoring system for spinal osteosynthesis, with connectors (2) allowing parallel connection rods (4, 5) to be fixed on each vertebral anchoring screw (3, 30), characterized in that each connector (2) comprises:
a first element (6) comprising parallel seats (8, 9) in the shape of a segment of a cylinder, each receiving the corresponding connection rod (4, 5), and a threaded hollow cylindrical sleeve (12) extending vertically between said seats (8, 9),
a second element (7) comprising parallel seats (10, 11) in the shape of a segment of a cylinder, each receiving the corresponding connection rod (4, 5), and a bore (25) arranged between said seats (10, 11) so as to be traversed by the cylindrical sleeve (12) of the first element (6),
a clamping nut (14) cooperating with an external thread (13) of the cylindrical sleeve (12) of the first element (6) in order, on the one hand, to fix the second element (7) on the first (6), and, on the other hand, to fix the connection rods (4, 5) in the seats (8, 9, 10, 11),
and clamping means (28, 32) cooperating with the cylindrical sleeve (12) of the first element (6) for fixing each connector (2) on the corresponding anchoring screw (3, 30).

2. The vertebral anchoring system for spinal osteosynthesis as claimed in claim 1, characterized in that the cylindrical sleeve (12) of the first element (6) of the connector (2) has, in its inner part, a through-bore (15) provided with a thread (16), a first surface (17) in the shape of a segment of a sphere, and a second surface (23) of conical profile.

3. The vertebral anchoring system for spinal osteosynthesis as claimed in claim 2, characterized in that the internal thread (16) of the cylindrical sleeve (12) cooperates with the clamping means (28) upon fixation of each connector (2) on a multi-axial anchoring screw (3).

4. The vertebral anchoring system for spinal osteosynthesis as claimed in claim 2, characterized in that the spherical surface (17) is continued, in the direction of the outside of the cylindrical sleeve (12), by the second surface (23) of conical profile whose widest base is oriented in the direction of the outside of the element (6).

5. The vertebral anchoring system for spinal osteosynthesis as claimed in claim 2, characterized in that the spherical surface (17) has, at its base, a shoulder (24) separating the latter from the conical surface (23), so as to constitute a support or bearing face for the corresponding fixation screw (3, 30).

6. The vertebral anchoring system for spinal osteosynthesis as claimed in claim 1, characterized in that the upper part, forming the free end of the cylindrical sleeve (12), has a plane face (31) forming a bearing face for the clamping means (32) upon fixation of each connector (2) on an anchoring screw with threaded head (30).

7. The vertebral anchoring system for spinal osteosynthesis as claimed in claim 1, characterized in that the cylindrical sleeve (12) of the first element (6) has, on its outer face and over its entire height, two parallel flat surfaces (18, 19).
